(19) United States
(12) Patent Application Publication
(10) Pub. No.: US 2017/0138967 A1
(43) Pub. Date: May 18, 2017

(54) METABOLICS BIOMARKER FOR NAFLD AND METHODS OF USE

(71) Applicant: Duke University, Durham, NC (US)

(72) Inventors: Manal F. Abdelmalek, Durham, NC (US); Anna M. Diehl, Durham, NC (US); Ricardo H. Giraldo, Durham, NC (US)

(21) Appl. No.: 15/356,050

(22) Filed: Nov. 18, 2016

Related U.S. Application Data
(60) Provisional application No. 62/256,760, filed on Nov. 18, 2015.
(60) Provisional application No. 62/378,305, filed on Aug. 23, 2016.

(51) Int. Cl. G01N 33/92; G01N 33/82; G01N 33/68

(52) U.S. Cl. CPC G01N 33/92; G01N 33/6893; G01N 33/82; G01N 2800/085; G01N 2800/56; G01N 2800/7052; G01N 2800/7085

(57) ABSTRACT

The present disclosure provides metabolomics biomarkers for NAFLD and indicative of liver fibrosis and methods of using same.

### CROSS-REFERENCE TO RELATED APPLICATIONS

This application claims priority to U.S. Provisional Application Nos. 62/256,760 filed on Nov. 18, 2015 and 62/378,305 filed on Aug. 23, 2016, the contents of which are incorporated by reference in their entireties.

### STATEMENT REGARDING FEDERALLY SPONSORED RESEARCH

N/A

### BACKGROUND OF THE INVENTION

Nonalcoholic fatty liver disease is currently the leading cause of chronic liver disease in the United States and estimated to affect nearly 100 million Americans. Approximately 25-30% of patients with NAFLD may develop nonalcoholic steatohepatitis (NASH) and be at risk for fibrosis progression. The gradual progression of fibrosis from mild to advanced stages is “silent” and may escape clinical detection by all other clinical, laboratory and/or radiologic testing. Short of a liver biopsy (which is associated with increased cost, health care utilization and potential risk), there is no noninvasive diagnostic test to stage NAFLD-related fibrosis which can be broadly utilized by any provider irrespective of their levels of expertise, training or scope of practice. The prevalence of nonalcoholic fatty liver disease (NAFLD) continues to increase with the growing obesity epidemic; NAFLD is now the leading cause of chronic liver disease in the Western World and in developing countries. NAFLD encompasses a histologic spectrum ranging from isolated hepatic steatosis to nonalcoholic steatohepatitis (NASH) characterized by lipid accumulation, inflammation, hepatocyte ballooning, and varying degrees of fibrosis. The severity of hepatic fibrosis is the primary predictor of increased morbidity and mortality in patients with NAFLD (FIG. 7). Although there is increasing public awareness for the risk of liver disease progression in patients with NASH, stratifying patients “at risk” for advanced hepatic fibrosis, and thus associated negative clinical outcomes, is hindered by the need for liver biopsy and the lack of non-invasive biomarkers. Discovery of a blood-based metabolomics biomarker would enable clinicians to stage hepatic fibrosis in patients with NAFLD to stratify risk and guide appropriate care and management.

Metabolomics is the scientific study of chemical processes involving metabolites. Specifically, metabolomics is the study of small-molecule metabolite profiles which may shed light on unique chemical “signatures” that are associated with specific cellular processes. The metabolome represents the collection of all metabolites in a biological cell, tissue, organ or organism, which are the end products of cellular processes. NAFLD is strongly associated with complex cellular processes including insulin resistance, diabetes mellitus, hyperlipidemia, hypertriglyceridemia, and overweight/obese status. Thus, NAFLD may be an ideal disease for which metabolic profiling can give an instantaneous snapshot of the disease physiology and severity. Previous work in a small cohort of NAFLD patient (n=25) demonstrated that metabolomics data could separate healthy subjects from NAFLD with an error rate of approximately 8% and separate NASH from healthy controls with an error rate of 4%. However, whether a metabolic profile differentiates NAFLD patients with hepatic fibrosis from those without fibrosis remains unknown. Unlike the low-risk cohort with mild hepatic fibrosis, NAFLD patients with advanced hepatic fibrosis require surveillance for complications of portal hypertension, screening for hepatocellular carcinoma and monitoring of liver synthetic function. However, the intermediate fibrosis group, for whom clinical risk and outcomes is not well defined, are typically followed and considered for repeat liver biopsies to restage fibrosis at intervals of 5-10 years. Non-invasive screening for advanced hepatic fibrosis could guide a clinician in diagnostic and therapeutic interventions, particularly for patients without the pathognomonic features of advanced liver disease.

There is a need to develop a biomarker profile that correlates with hepatic fibrosis in patients suffering from NAFLD.

### SUMMARY OF THE INVENTION

The present disclosure provides metabolomics biomarkers for nonalcoholic fatty liver disease (NAFLD) and methods of using same. The biomarkers are able to predict, prognose and/or diagnose the severity of liver fibrosis.

Another aspect of the present disclosure provides all that is described and illustrated herein.

In one aspect, the disclosure provides a panel of non-invasive metabolomic biomarkers for nonalcoholic fatty liver disease (NAFLD) comprising at least 2, at least 3, at least 4, or at least 10 metabolomic biomarkers listed in any of Table 2, Table 7, Table 8 or Table 9 which are associated with NFLD. In some aspects, the panel of metabolomic biomarkers are associated with liver fibrosis, specifically associated with severity of liver fibrosis in a subject.

In another aspect, the disclosure provides a panel of metabolomic biomarkers comprising taurochenodeoxycholate, glyocholate, glycochenodeoxcholate, and taurocholate, wherein the expression of the panel is associated with advanced hepatic fibrosis (stage 3-4).

In another aspect, the disclosure provides a panel of metabolomic biomarkers comprising glutamate, 3 methlglutarylcarintine and glutamate, and wherein the expression of the panel is associated with advanced hepatic fibrosis.

In yet another aspect, the disclosure provides a method of detecting a panel of metabolomic biomarkers associated with nonalcoholic fatty liver disease or liver fibrosis in a subject. The method comprises obtaining a sample from a subject; detecting at least 2, at least 3, at least 4, or at least 10 non-invasive metabolite fibrosis markers listed in any one of Tables 2, 7, 8, or 9 in a sample obtained from the subject; wherein the detection of at least two markers is associated with NAFLD or liver fibrosis. In one embodiment, the sample is a serum sample or blood sample.

In some aspects, the expression of at least three, at least four, at least five, at least six, at least seven, at least eight, at least nine, at least 10, at least 11, at least 12, at least 13, at least 14 metabolomics biomarkers are detected.

In some further aspects, the method further comprises diagnosing the patient with liver fibrosis. The method allows for the diagnosis without a liver biopsy or other invasive technique.

In yet another aspect, a method of diagnosing or prognosing liver fibrosis in a subject, wherein the method comprises obtaining a sample from a subject, detecting at least 2, at least 3, at least 4, or at least 10 non-invasive metabolomic biomarkers listed in any one of Tables 2, 7, 8, or 9 in a sample obtained from the subject; wherein the detection of at least two metabolomic biomarkers is associated with liver fibrosis. In some embodiments, the metabolomic biomarkers are associated with the severity of liver fibrosis. In some embodiments, the higher expression of the metabolomic biomarkers is associated with increase severity, e.g. advanced stage, of liver fibrosis. In some embodiments, the method further comprises treating the subject diagnosed or prognosed with liver fibrosis.

In yet another aspect, the invention provides a kit for detecting NAFLD or liver fibrosis in a subject comprising means for detecting at least 2, at least 3, at least 4, or at least 10 metabolomic biomarkers in a sample.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A. Elevations in bile acids were observed in patients with advanced liver fibrosis (top left). Primary bile acid pathway (top right) hierarchical clustering of observed primary bile acid metabolites shows agreement with the known pathway.

FIG. 1B. Log-expression values of primary bile acid metabolites grouped by fibrosis stage. Accumulation of bile acids may be attributable to a combination of increased synthesis and decreased hepatic function (extraction).

FIG. 1C. Log-expression values of primary bile acid metabolites grouped by fibrosis stage. Accumulation of bile acids may be attributable to a combination of increased synthesis and decreased hepatic function (extraction).

FIG. 2A. Accumulation of various TCA cycle intermediates was observed in patients with stage 3-4 fibrosis.

FIG. 2B. TCA cycle pathway hierarchical clustering of observed metabolites in TCA cycle shows agreement with the TCA cycle pathway.

FIG. 2C. Log-expression values of TCA cycle metabolites grouped by fibrosis stage. Increased levels of TCA cycle intermediates has been traditionally associated with decreased mitochondrial oxidative activity and energy generation, and may be reflective of mitochondrial dysfunction in patients with advanced fibrosis.

FIG. 3. ROC curves for 3 different fibrosis strata: (left) B 1: mild vs. advanced, (middle) B2: mild/intermediate vs. advanced, and (right) B3: mild vs. intermediate/advanced. AUC values for each ROC curve are shown in Table 3. We consider seven models: five published non-invasive markers (AST/ALT ratio, BARD score, Fib4 score, APRI index, NAFLD fibrosis score, NAFLD clinical score) and the two proposed models, metabolomics binary and metabolomics ordinal, based on sparse logistic regression and ordinal logistic regression, respectively.

FIG. 4. Sensitivity and specificity: (left) Sensitivity (TPR) and specificity (TNR) vs. decision threshold for the ordinal model and B3 comparison. (Right) Threshold values with corresponding TPR and TNR values.

FIG. 5. Chart depicting the correlation of risk and fibrosis as the primary predictor of negative clinical outcome.

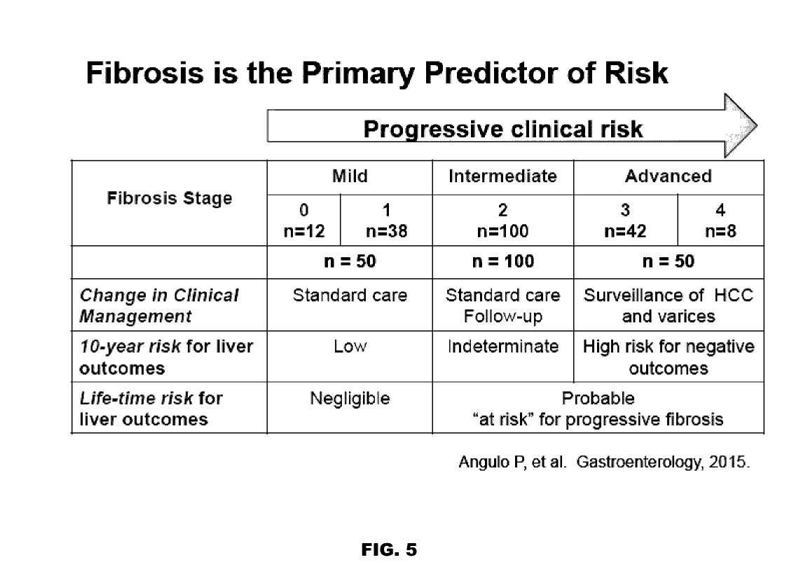

FIG. 6. ROC curves for F0-2 vs 3-4 strata. Six models were compared, five published non-invasive markers (AST/ALT ratio, BARD score, Fib4 score, APRI index, NAFLD fibrosis score, NAFLD clinical score) and metabolomics model.

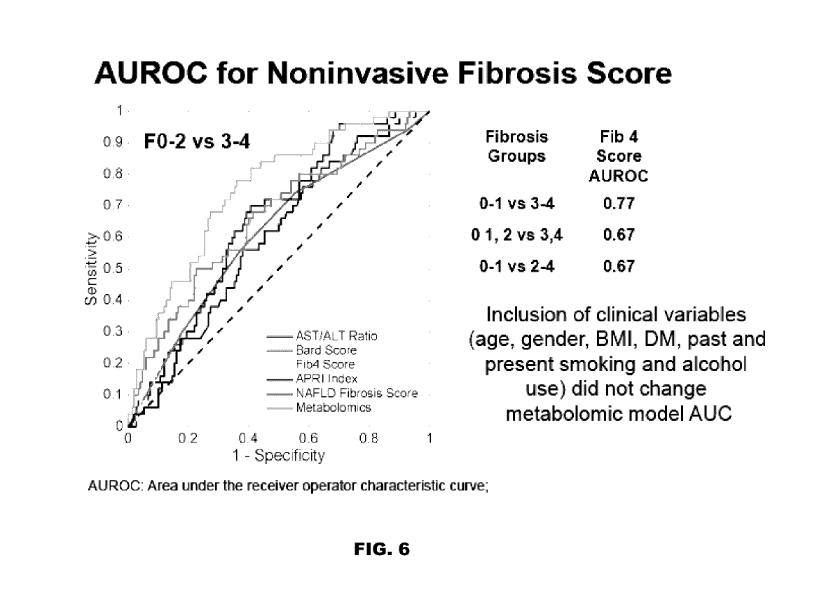

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

As used herein the term patient and subject can be used interchangeably. The patient or subject is suitably a mammal, more preferably a human. In some embodiments, the metabolomics biomarkers and methods may be used to diagnose, prognose or treat a mammal, for example, a human, a chimpanzee, a mouse, a rat, a dog, a cat, a horse or other livestock. In the most preferably embodiment, the method is used to diagnose, prognose or treat a human.

Unless otherwise defined, all technical terms used herein have the same meaning as commonly understood by one of ordinary skill in the art to which this disclosure belongs.

Severity of hepatic fibrosis strongly predicts liver-related morbidity and mortality for patients with nonalcoholic fatty liver disease (NAFLD). Unfortunately, noninvasive biomarkers for stratifying advanced hepatic fibrosis in NAFLD are currently lacking. We analyzed the metabolic alterations patients with biopsy-proven NAFLD (n=200 serum and 20 paired plasma samples) from our Duke NAFLD Clinical Database and Biorepository to assess the predictive metabolic profile of hepatic fibrosis. Patients were stratified based on fibrosis severity: mild, stages 0-1 (n=50); intermediate, stage 2 (n=100); and advanced, stages 3-4 (n=150). Metabolomic analysis was performed using ultrahigh performance liquid chromatography/tandem mass spectrometry (UHPLC/MS/MS) optimized for basic and acidic species and gas chromatography/mass spectrometry. Generalized linear regression while controlling for several clinical variables was used to correlate metabolites with fibrosis stage. Binary classification via sparse logistic regression of several stratifications (mild, intermediate and advanced fibrosis) yielded good leave-one-out cross-validation area under the curve (AUC). Ordinal regression yielded the best correlation with fibrosis stage and overall performance measures (accuracy, sensitivity, specificity and AUC). Binary classification results compared favorably with existing non-invasive markers for fibrosis (AUC 0.864 for the mild vs. advanced comparison). Our analysis identified a metabolic profile of 84 metabolites based on ordinal regression, which are associated with severity of hepatic fibrosis. Such metabolites are primary associated with bile acid metabolism, TCA cycle, and amino acid metabolism. This metabolic profile, after controlling for potential confounding variables, accurately stratified fibrosis severity even after correcting for multiple comparisons. Conclusions: Metabolomics can serve as a sensitive and specific biomarker to stratify fibrosis severity in patients with NAFLD and has potential to stratify patients at highest risk for liver-related clinical outcomes.

The present disclosure provides noninvasive biomarkers for stratifying advanced hepatic fibrosis in NAFLD and methods of detecting, prognosing and treating hepatic fibrosis in NAFLD.

In some embodiments, the present invention provides a panel of non-invasive metabolomic biomarkers for nonalcoholic fatty liver disease (NAFLD) comprising at least 2, at least 3, at least 4, or at least 10 metabolomic biomarkers listed in any of Table 2, Table 7, Table 8 or Table 9 which are associated with NFLD. In some embodiments, the detection of the panel of metabolomics biomarkers indicates or is associated with liver fibrosis. Increased levels of the metabolomics biomarkers is associated with increased severity of liver fibrosis.

The severity of liver fibrosis is categorized into mild (stage 0-1) which confers low risk, moderate (stage 2) which confers indeterminate risk; or advanced (stage 3 or 4) which confers high risk for liver-related outcomes over 10 years. Increased severity of liver fibrosis is associated with increased negative outcomes for the patient and increased complications of liver disease and fibrosis.

In some embodiments, methods of determining the severity of liver fibrosis are provided in patients with NAFLD. Methods of determining the risk of increased negative outcomes for patients with NAFLD, liver disease or fibrosis are also provided.

Suitably, the metabolomic biomarkers are selected from the group consisting of the biomarkers listed in Table 1, 7, 8 or 9.

In one embodiment, the panel of metabolomic biomarkers are two or more, 3 or more, 4 or more, 5 or more, 6 or more, 7 or more, 8 or more, 9 or more, 10 or more, 11 or more, 12 or more, 13 or more, 14 or more biomarkers selected from the biomarkers listed in Table 1, 7, 8 or 9. In one embodiment, the biomarkers include two or more, 3 or more, 4 or more, 5 or more, 6 or more, 7 or more, 8 or more, 9 or more, 10 or more, 11 or more, 12 or more, 13 or more, 14 or more biomarkers listed in Table 2. In another embodiment, .the panel of biomarkers include the two or more, 3 or more, 4 or more, 5 or more, 6 or more, 7 or more, 8 or more, 9 or more, 10 or more, 11 or more, 12 or more, 13 or more, 14 or more biomarkers in Table 7. In another embodiment, .the panel of biomarkers include the two or more, 3 or more, 4 or more, 5 or more, 6 or more, 7 or more, 8 or more, 9 or more, 10 or more, 11 or more, 12 or more, 13 or more, 14 or more biomarkers in Table 8. In another embodiment, .the panel of biomarkers include the two or more, 3 or more, 4 or more, 5 or more, 6 or more, 7 or more, 8 or more, 9 or more, 10 or more, 11 or more, 12 or more, 13 or more, 14 or more biomarkers in Table 9.

In some embodiments, the panel of biomarkers comprises taurochenodeoxycholate, glyocholate, glycochenodeoxcholate, and taurocholate, wherein the expression of the panel is associated with advanced hepatic fibrosis (stage 3-4). In another embodiment the panel of biomarkers comprises glutamate, 3 methlglutarylcarintine and glutamate, and wherein the expression of the panel is associated with advanced hepatic fibrosis.

Methods of detecting a panel of metabolomics biomarkers associated with nonalcoholic fatty liver disease or liver fibrosis in a subject are provided. The method comprises obtaining a sample from a subject; detecting at least 2, at least 3, at least 4, or at least 10 non-invasive metabolite fibrosis markers listed in any of Tables 2, 7, 8, or 9 in a sample obtained from the subject; wherein the detection of at least two markers is associated with NAFLD or liver fibrosis.

The term sample refers to a biological sample obtained from a patient. In the preferred embodiment, the biological sample is serum or a blood sample obtained from the patient. In a suitable embodiment, the sample is a blood sample obtained from a fasting patient.

In some methods, the expression of at least three, at least four, at least five, at least six, at least seven, at least eight, at least nine, at least 10, at least 11, at least 12, at least 13, at least 14 metabolomics biomarkers are detected.

The expression of the metabolomic biomarkers indicates the presence of NAFLD or liver fibrosis in the patient. An increased level of the metabolomic biomarkers is indicative and associated with an increased severity or higher grade liver fibrosis.

In some embodiments, the method of detecting at least one metabolomic biomarker further comprises diagnosing or prognosing the patient with liver fibrosis. Increased level of the at least two metabolomic biomarkers indicates a more advanced level or stage of fibrosis.

The methods may further comprise treating the subject diagnosed or prognosed with liber fibrosis. Suitable methods of treating fibrosis are known in the art and include, for example, administering an anti-fibrotic therapy to the subject diagnosed or prognosed with liver fibrosis. Suitable anti-fibrotic therapy are known in the art and include, but are not limited to, angiotensin inhibitors, colchicine, corticosteroids, endothelin inhibitors, interferon-alpha, interleukin-10, pentoxyfylline, phosphatidylcholine, PPAR antagonists, S-adenosyl methionine, Sho-saiko-to, TGF-β1inhibitors, Tocopherol and the like. The anti-fibrotic therapy may be administered orally or via intravenous administration.

In one embodiment, the metabolite markers comprise taurochenodeoxycholate, glyocholate, glycochenodeoxcholate, and taurocholate, and wherein the expression of the panel is associated with advanced hepatic fibrosis (stage 3-4). In another embodiment, the metabolite markers comprise glutamate, 3 methlglutarylcarintine and glutamate or malate and fumarate, and the expression of the markers is associated with advanced hepatic fibrosis.

In some embodiments, kits for carrying out the methods of the present disclosure are provided. For example, a kit for detecting one or more metabolomic biomarkers is provided. Further kits for detecting and treating liver fibrosis are provided. The kit comprises means for detecting the one or more metabolomic biomarker. Suitable means for detecting the biomarker are known in the art and include, but are not limited to, reagents such as PCR primers, antibodies. Instructions may also be provided. In one embodiment, a kit for detecting NAFLD or liver fibrosis in a subject comprising means for detecting at least two, at least 3, at least 4, or at least 10 metabolomic biomarkers in a sample are provided.

By “treating” or “treatment,” we mean the management and care of a subject for the purpose of combating and reducing liver disease. Treating may reduce, inhibit, ameliorate and/or improve the onset of the symptoms or complications, alleviating the symptoms or complications of the tumor, or eliminating liver disease or liver fibrosis. As used herein, the term “treatment” is not necessarily meant to imply cure or complete abolition of the liver disease. Treatment may refer to the inhibiting or slowing of the progression of the liver disease or liver fibrosis, reducing the incidence of liver fibrosis, or preventing additional progression of liver fibrosis.

By “ameliorate,” “amelioration,” “improvement” or the like we mean a detectable improvement or a detectable change consistent with improvement occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range about between any two of these values. Such improvement or change may be observed in treated subjects as compared to subjects not treated.

The following non-limiting examples are included for purposes of illustration only, and are not intended to limit the scope of the range of techniques and protocols in which the compositions and methods of the present invention may find utility, as will be appreciated by one of skill in the art and can be readily implemented.

### EXAMPLE 1

Plasma Metabolic Profiles Predict Liver Fibrosis

### A. Materials and Methods

Human Subjects Cohort

We conducted a cross-sectional study utilizing prospectively collected data from NAFLD subjects in the Duke University Health System (DUHS) NAFLD Clinical Database and Biorepository. The DUHS NAFLD Clinical Database and Biorepository is approved by our Institutional Review Board and contains clinical data, serum, plasma, and frozen liver tissue from NAFLD patients who underwent a diagnostic liver biopsy to grade and stage severity of disease as part of standard of care. Biospecimens are collected at the time of standard of care liver biopsy and after a 12 hour fast for the scheduled procedure. Patient reported data was obtained via self-reported questionnaires administered on the day of liver biopsy and/or systematic chart review and data extraction. Only patients who consented to utilize their samples for “—omics” analysis were included in our analysis.

For the present study, NAFLD was defined as: (1) presence of >5% hepatic steatosis on liver biopsy, (2) absence of histologic and serologic evidence for other forms of chronic liver disease, and (3) little or no alcohol consumption (<20 g/day for women and <30 g/day for men). Demographic and clinical data evaluated included height, weight, body mass index (BMI, kg/m2), age, gender, race, ethnicity, smoking status, social alcohol use, fasting lipid profiles [total cholesterol, triglycerides, high density lipoprotein cholesterol (HDL-C), and low density lipoprotein cholesterol (LDL-C)], as well as the presence of hypertension, diabetes mellitus (DM), hyperlipidemia and dyslipidemia were obtained at the time of liver biopsy. BMI was calculated using the formula: weight (in kilograms)/height (in meters). Obesity was diagnosed when BMI was ≧30 kg/m², and overweight when BMI was ≧25 and <30 kg/m². Laboratory studies (i.e. lipids, glucose, liver aminotransferases, and measures of liver synthetic function) were obtained within 6 months of liver biopsy on all patients. The presence of hypertension was defined by known diagnosis in the medical record and/or the use of any antihypertensive medication. The presence of DM was defined by known diagnosis in the medical record, use of any insulin-sensitizing agent or insulin, and/or a glycosylated hemoglobin (HbA1c) value of >6.5%. Hyperlipidemia was defined as a total cholesterol level >200 mg/dl, an LDL-C >120 mg/dl and/or use of a lipid lowering agent. Smoking and social alcohol use were defined as yes or no if a patient was a smoker or social user of alcohol at the time of liver biopsy.

The study cohort was selected for inclusion into this analysis based on severity of hepatic fibrosis, a key determinant of clinical outcome. Patients were matched across fibrosis stages for gender, age (+/−5 years) and BMI (+/−3 points). All liver biopsies were graded and staged according to the Steatohepatitis Clinical Research Network. The study cohort was divided into three groups based on the severity of hepatic fibrosis: 1) mild fibrosis (stage 0-1; n=50) which confers low risk; 2) intermediate fibrosis (stage 2; n=100) which confers indeterminate risk; and 3) advanced fibrosis (stage 3-4; n−50) which confers high risk for liver-related outcomes over 10 years.

The clinical patient characteristics are listed in Table 4.

Liver Histology

The primary outcome measure was fibrosis stage on liver biopsy. All liver biopsy specimens were stained with hematoxylin-eosin and Masson' s trichrome stains, and reviewed and scored according to the published NASH Clinical Research Network scoring system. For the analyses, fibrosis stages 1a, 1b, and 1c were combined and treated as stage 1 fibrosis. Portal inflammation was scored as 0=absent, 1=present. The histological patient characteristics outlined in Table 5.

Metabolomics Platform

Sample Handling and Processing. All samples were maintained at −80° C. until processed on the metabolomics platform of Metabolon, Inc. (Durham, N.C.). Samples were extracted and split into equal parts for analysis on the gas chromatography/mass spectrometry (GC/MS) and liquid chromatography/tandem mass spectrometry (LC/MS/MS) platforms. A combination of three platforms was used for metabolic profiling: Ultrahigh performance Liquid Chromatography/tandem Mass Spectrometry (UHPLC/MS/MS) optimized for basic species, UPHLC/MS/MS optimized for acidic species, and GC/MS as previously described.

GC/MS and UHPLC/MS analysis. UHPLC/MS was performed using a Waters Acquity UHPLC (Waters Corporation, Milford, Mass.) coupled to a linear trap quadrupole (LTQ) mass spectrometer (Thermo Fisher Scientific Inc., Waltham, Mass.) equipped with an electrospray ionization source. Two separate UHPLC/MS injections were performed on each sample: one optimized for positive ions and one for negative ions. Samples for GC/MS were analyzed on a Thermo-Finnigan Trace DSQ fast-scanning single-quadrupole MS operated at unit mass resolving power. Chromatographic separation followed by full scan mass spectra were carried out to record retention time, molecular weight (m/z) and MS/MS of all detectable ions presented in the samples.

Lipidomics Panel. Lipids were extracted in the presence of authentic internal standards using chloroform:methanol. Lipids were trans-esterified in 1% sulfuric acid in methanol in a sealed vial under a nitrogen atmosphere at 100° C. for 45 minutes. The resulting fatty acid methyl esters were extracted from the mixture with hexane containing 0.05% butylated hydroxytoluene and prepared for GC by sealing the hexane extracts under nitrogen. Fatty acid methyl esters were separated and quantified by capillary GC (Agilent Technologies 6890 Series GC) equipped with a 30 m DB 88 capillary column (Agilent Technologies) and a flame ionization detector.

Quality Assurance and Control. Several types of controls were analyzed in concert with the experimental samples. Specifically, a pooled matrix sample generated by taking a small volume of each experimental sample (or alternatively, use of a pool of well-characterized human plasma) served as a technical replicate throughout the data set. Extracted water samples served as process blanks and a cocktail of quality control (QC) standards were chosen not to interfere with the measurement of endogenous compounds were spiked into every analyzed sample, allowed instrument performance monitoring and aided chromatographic alignment. Instrument variability was determined by calculating the median relative standard deviation (RSD) for the standards that were added to each sample prior to injection into the mass spectrometers. Process variability was determined by calculating the median RSD for all endogenous metabolites (i.e., non-instrument standards) present in 100% of the pooled matrix samples. Experimental samples were randomized across the platform run with QC samples spaced evenly among the injections.

Metabolite Identification. Metabolites were identified by automated comparison of the ion features in the experimental samples to a reference library of chemical standard entries that included retention time, molecular weight (m/z), preferred adducts, and in-source fragments as well as their associated MS/MS spectra. This library allowed the rapid identification of metabolites in the experimental samples with high confidence. The metabolomics data yielded 1151 (n=790 identified and 361 unidentified) metabolites for analysis from 8 super-pathways: amino acids (n=157), peptides (n=55), carbohydrates (n=28), energy related (n=7), lipids (n=307), nucleotides (n=34), cofactors/vitamins (n=34) and xenobiotics (n=167) (Table 6).

Assessment of Other Non-invasive Determinants of Fibrosis Markers

The aspartate aminotransferase (AST) to alanine aminotransferase (ALT) ratio, AST to Platelet Ratio Index (APRI), Fibrosis-4 (FIB-4) score, BARD score and NAFLD fibrosis scores were calculated for each patient. The APRI index was determined according to Wai et al. as follows: (actual AST concentration divided by its upper normal limit)/platelet counts (10⁹/L)×100. The FIB-4 score was determined according to Sterling et al (10) as follows: age (years)×AST[U/L]/(platelets[10⁹/L]×(ALT[U/L])^{1/2}). The NAFLD fibrosis score formula is −1.675+0.037×age (years)+0.094×BMI (kg/m²)+1.13×IFG/diabetes (yes=1, no=0)+0.99 ×AST/ALT ratio−0.013×platelet count (×10⁹/l)−0.66×albumin (g/dl). For calculation of NAFLD fibrosis score, diabetes mellitus was defined as fasting glucose was >126 mg/dl or use of anti-diabetic drugs, and impaired fasting glucose (IFG) was defined as fasting glucose level between 100 and 125 mg/dl. According to Angulo et al, a score lower than —1.455 (low cutoff) excludes advanced fibrosis, whereas a score higher than 0.676 (high cutoff) predicts advanced fibrosis. Scores between these values are indeterminate. The BARD score is composed of 3 variables: AST/ALT ratio>0.8=2 points; a BMI>28=1 point; and the presence of diabetes =1 point. The possible score ranges from 0 to 4 points. According to the results of Harrison et al., BARD scores equaling 0 or 1 are high predictive of the absence of advanced fibrosis (12). The NAFLD Clinical Score for Advanced Fibrosis was calculated as −11.8+0.04×age (years)+0.042×BMI (kg/m²)+3.05 ×waist-to-hip ratio+0.014×alkaline phosphatase (ALK) (units/L)+1.26×AST/ALT ratio+1.23×albumin (g/dL)+0.82×globulin (g/dL)−0.103×hematocrit (%)−0.0133×platelet count (1,000/mm³)+3.19×direct bilirubin (mg/dL)−0.81×total bilirubin (mg/dL)−1.33×if abnormal ALK +1.56×INR+0.0131×serum insulin (μU/ml)−0.79×if Hispanic+0.44 if hypertensive.

B. Statistical Analysis

We excluded 361 (of 1151) unidentified serum metabolites from our analysis. Metabolites for which there was more than 50% missing values (106 of 789 identified metabolites), were also excluded from the analysis. For metabolites with less than 50% missing values, these were values imputed to half of the observed minimum value for any given metabolite. Data was log-transformed prior to statistical analysis. Minor technical variations resulting from instrument inter-day differences (i.e., batch effects) were corrected by removing mean day-wise effects from each analyte independently after log-transformation.

Univariate analysis. Univariate testing to compare fibrosis stage with demographics, laboratory data and metabolomics were performed using one-way Analysis of Variance (ANOVA) with False Discovery Rate (FDR)-adjusted p-values to control for multiple testing. In all analyses we considered adjusted p-values <0.05 to be significant. The top 10 of 790 metabolites with fibrosis stage normalized for run day only are listed in Table 7.

Multivariate analysis. Generalized linear regression was used to assess metabolite associations with fibrosis stage while controlling for different groups of possible confounders. We also employed two sparse models. Inherent in their construction is a concept called “variable selection.” Variable selection automatically identifies covariates that are most likely to impact predictive power; thus sparse models have the desirable property of enabling prediction, while at the same time selecting for the metabolites with the greatest predictive power.

First, Sparse logistic regression (Least Absolute Shrinkage and Selection Operator—LASSO), was used as a classification model for different binary partitions of fibrosis stage (e.g., 0,1,2, vs. 3,4). Second, we directly modeled fibrosis stage as an ordinal variable using sparse ordinal regression. Similar to sparse logistic regression in its usage of a link function (logistic), ordinal regression utilizes the rank-likelihood link, to relate the multivariate regression function, f(x), to the target variable (fibrosis stage), where f(x) is a weighted combination of metabolite measurements, x, were used. Performance of these models was quantified via the Receiving Operating Characteristics (ROC) curve and Area Under the ROC Curve (AUC). ROC curves were computed using the regression function,f(x). Statistical validation was done via nested leave-one-out cross-validation to select model parameters, correct for batch effects and estimate ROC and AUC values in an unbiased manner. Statistical analyses were performed in MATLAB 2015a (The MathWorks, Inc., Natick, Mass., USA).

C. Results

Patient Characteristics

Demographic and clinical data obtained from 200 study participants are summarized in Table 1. Our cohort was predominantly Caucasian (89%) and obese (BMI 35 ±7.2 kg/m²) with a substantial minority having diabetes mellitus (37.5%). The diagnosis of diabetes mellitus increased with increasing fibrosis stage (p<4.9×10⁻⁵) as was also evidenced by the strong association between increased glycosylated hemoglobin (p<2.4×10⁻³) and increased fibrosis stages. Likewise, a mild increase in total bilirubin (p<0.029) and decline in platelet count (p<8.9×10⁻⁴) was associated with increased fibrosis stages. No difference in batch effects, age, gender, BMI, total cholesterol, LDL-C, HDL-C, triglycerides, AST, ALT, past or present alcohol or tobacco use and/or hepatic steatosis were noted across fibrosis stages. As anticipated, portal inflammation, lobular inflammation and hepatocyte ballooning increased with increased fibrosis stages (p=9.6×10⁻⁶, p=1.3×10⁻³, and p=2.9×10⁻⁷, respectively).

Univariate Analysis

Results of the univariate analysis with metabolites, the pathway to which they belong, and the statistical significance based on the analytical model performed are detailed in Table 2 and Table 7. The univariate analysis was performed using 4 models. Model 1 (M1) controlled only for batch effects (i.e., differences that may have occurred simply because samples were analyzed on different days). Model 2 (M2) controlled for batch effect plus age, gender, BMI, and past and present alcohol consumption, as well as past and present smoking. Model 3 (M3) included all the variables in model 2 plus the known diagnoses of diabetes mellitus and hypertension. Model 4 (M4) included model 3 plus histologic features of steatosis, lobular inflammation, portal inflammation, and ballooned hepatocytes. Model 4 (results not shown but noted in Supplemental Table 1) revealed no significant differences when compared to model 3 thereby indicating that the significant metabolites noted in Model 3 were not confounded or altered in their significance by histologic features other than fibrosis.

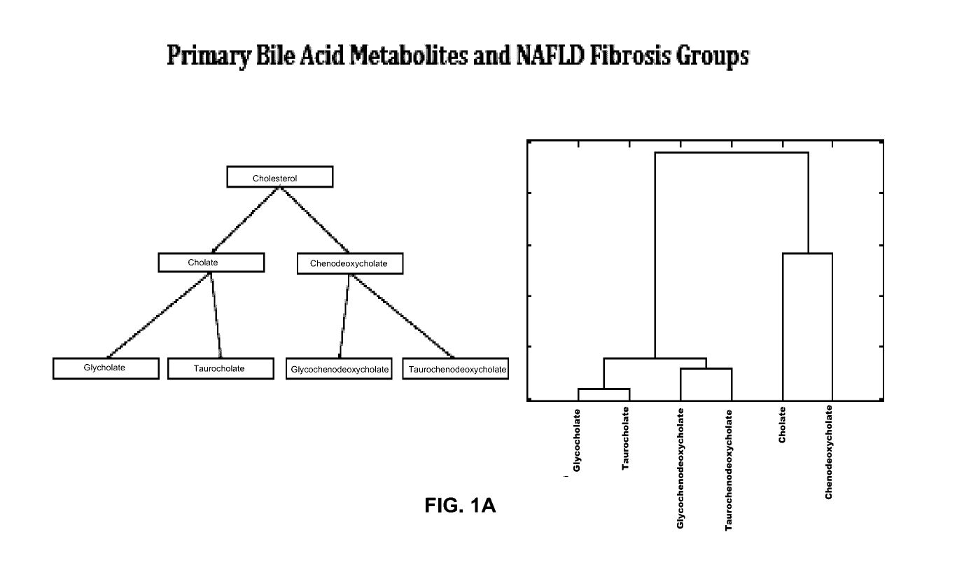

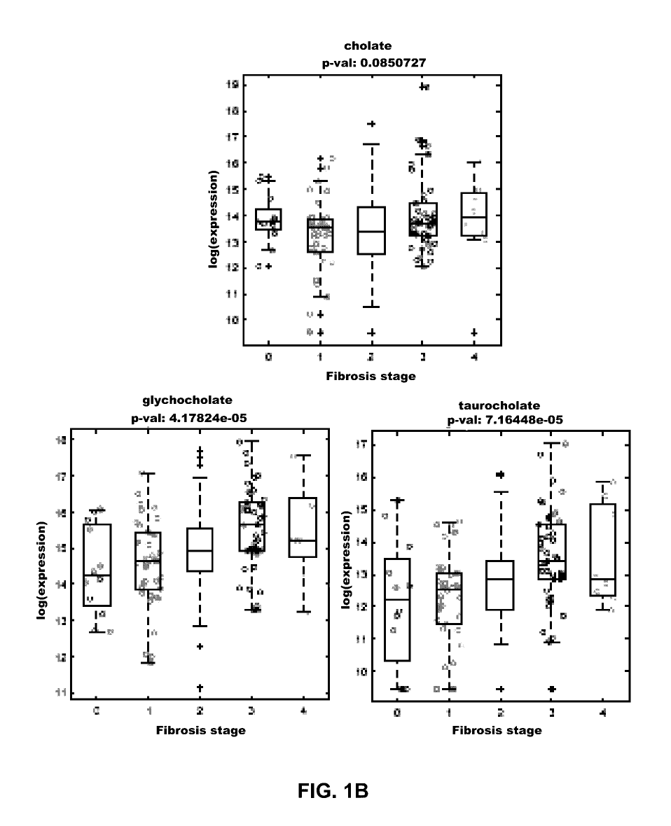

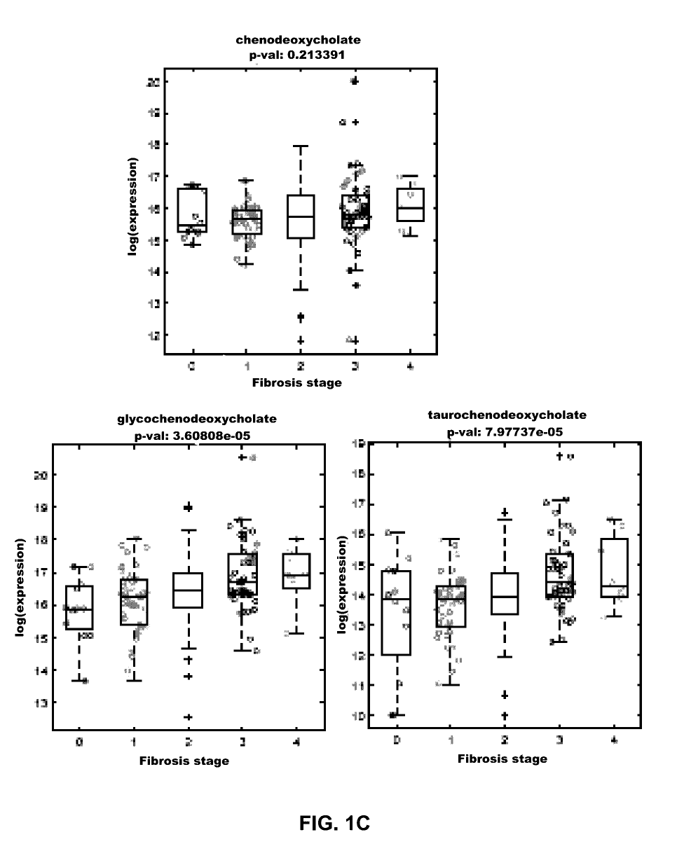

Of interest, 4 of the top 10 metabolites that correlated with liver fibrosis severity belonged to the pathway of primary bile acid metabolism. Specifically, taurochenodeoxycholate, glyocholate, glycochenodeoxcholate, and taurocholate were associated with advanced hepatic fibrosis (stage F3-4) in all three models (p<0.05 for M1, M2 and M3), and remained significant after controlling potential confounding factors which were included in the analytical models. The association taurochenodeoxycholate, glyocholate, glycochenodeoxcholate, and taurocholate and fibrosis severity further detailed scatter plots in FIG. 1.

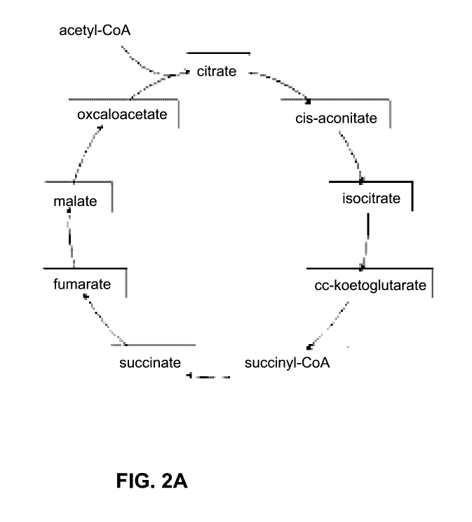

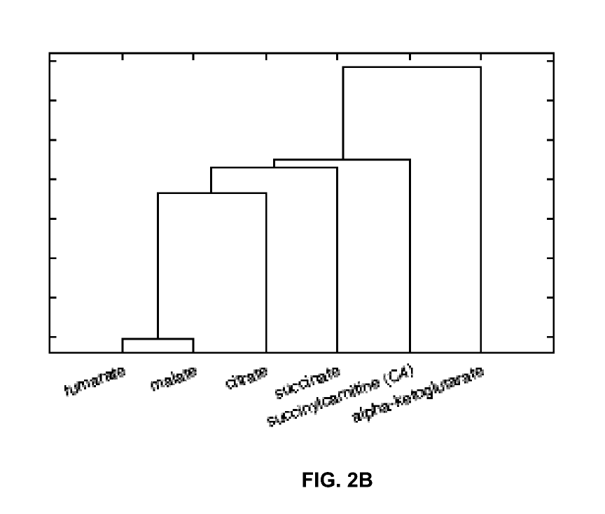

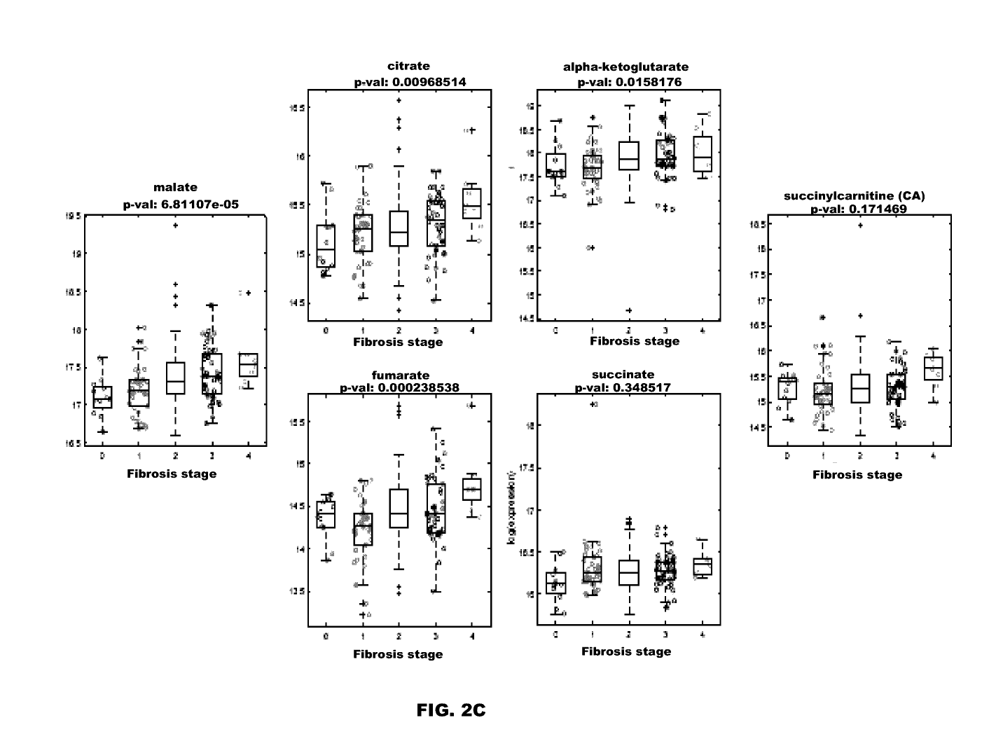

Additional changes in metabolites (malate and fumarate) reflective of perturbations in energy metabolism and decreased mitochondrial function was observed in patients with stage 3-4 fibrosis. This is plausible, as mitochondrial dysfunction has been implicated in the pathogenesis of NAFLD and NASH, as well as conditions associated with metabolic syndrome that often accompany fatty liver disease. The accumulation of tricarboxylic acid cycle (TCA) cycle intermediates has traditionally been associated with decreased mitochondrial oxidative activity and energy generation. The alterations in TCA cycle intermediates in NAFLD fibrosis groups were increased in our study and detailed in FIG. 2. Lastly, univariate analysis revealed increases in branched-chain amino acids (BCAA), specifically glutamate, 3-methlglutarylcarintine and glutamate in patients with advanced hepatic fibrosis.

Multivariate Analysis

We built sparse logistic regression models based on metabolic data to predict three distinct binary stratifications of fibrosis stage, namely, mild vs. advanced (B1: stage 0,1 vs. 3,4), mild/intermediate vs. advanced (B2: stage 0,1,2 vs. 3,4) and mild vs. intermediate/advanced (B3: stage 0,1 vs. 2,3,4). These specific strata were based on predictors which:

(1) influence clinical management for screening of hepatocellular carcinoma and endoscopic screening for esophageal varices in patients with advanced hepatic fibrosis (18, 19),

(2) define the future risk for hepatic decomposition (20) and

(3) constitute a potential biomarker to define those at increased risk for all-cause and liver-related morbidity and mortality (21-23) in patients with NAFLD.

We also considered a single sparse ordinal model to predict fibrosis stage directly, that is, an ordinal variable with values 0, 1, 2, 3 and 4. Sparse ordinal models are beneficial because they forgo the need to stratify fibrosis stage a priori, as is traditionally done in models specified for binary fibrosis strata (i.e., B1, B2 and B3). Examples of these binary models, all based on logistic regression are: APRI index, FIB-4, NAFLD fibrosis score and NAFLD clinical score. Modeling fibrosis stage directly via ordinal models is beneficial because it helps to identify metabolites (signatures) that correlate with fibrosis stage. This is not the case for logistic regression where metabolites are identified by their ability to discriminate fibrosis stage subgroups, e.g. 0,1,2 vs. 3,4, rather than the complete spectrum of fibrosis stage.

Sparse ordinal models do not stratify fibrosis a priori, however, can be performed a posteriori to gain insight about the performance characteristics of the model, or to compare the different models. In particular, below we report leave-one-out cross-validated AUC values as performance metric for the three previously defined binary stratifications of fibrosis.

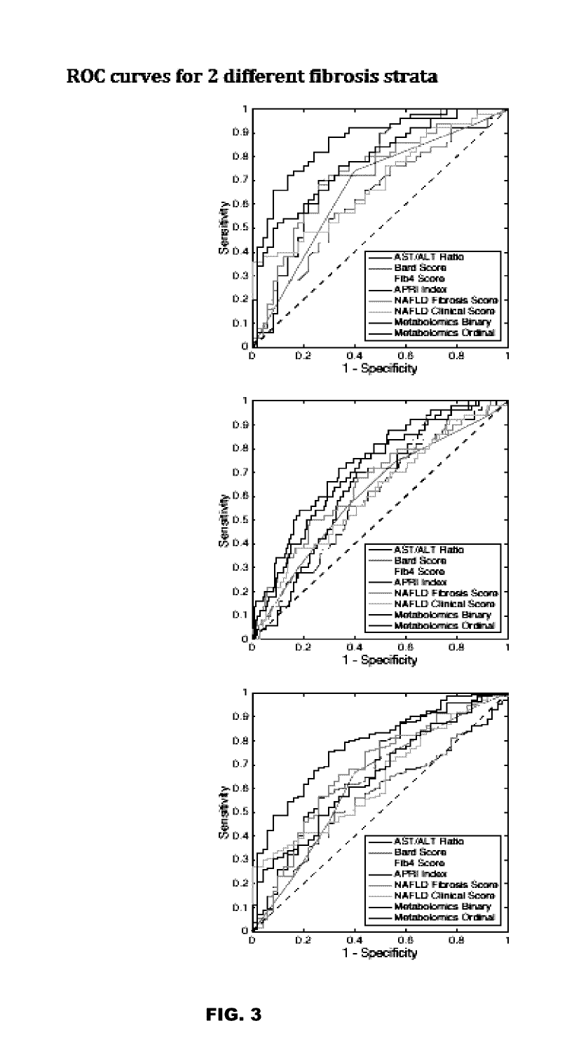

We compared our metabolomics-based predictors (logistic and ordinal) against 6 published non-invasive markers of fibrosis, namely, AST/ALT ratio, BARD score, APRI index, FIB-4 score, NAFLD fibrosis score and NAFLD Clinical Score. Results in Table 3 show that our models, based purely on metabolic measurements, outperform all the other markers in the three tasks considered. We found that the ordinal model has the best performance. FIG. 3 and FIG. 8 shows ROC curves for the mild vs. advance comparison (B1), mild/intermediate vs. advanced (B2), and mild vs. intermediate/advanced (B3). The metabolic signatures identified by our models as being predictive of fibrosis consist of 33, 83 and 76 metabolites for sparse logistic regression based B 1, B2 and B3, respectively, and 83 metabolites for the ordinal model. There were 14 metabolites common to all models (B1, B2, B3 and ordinal), from which 33 are in at least 3 models and 134 in any model. Common metabolites include dehydroisoandrosterone sulfate, theobromine, 16a-hydroxy DHEA 3-sulfate, N-acetyl-1-methylhistidine, erucic acid, inosine, N-acetylmethionine, glycochenodeoxycholate. Only glycochenodeoxycholate was identified as relevant in the univariate analysis after correction for multiple testing. Sparse models are known to favor selecting fewer metabolites by excluding strongly correlated metabolites; therefore, the lack of overlap in reported metabolites in the univariate and multivariate analysis is not surprising.

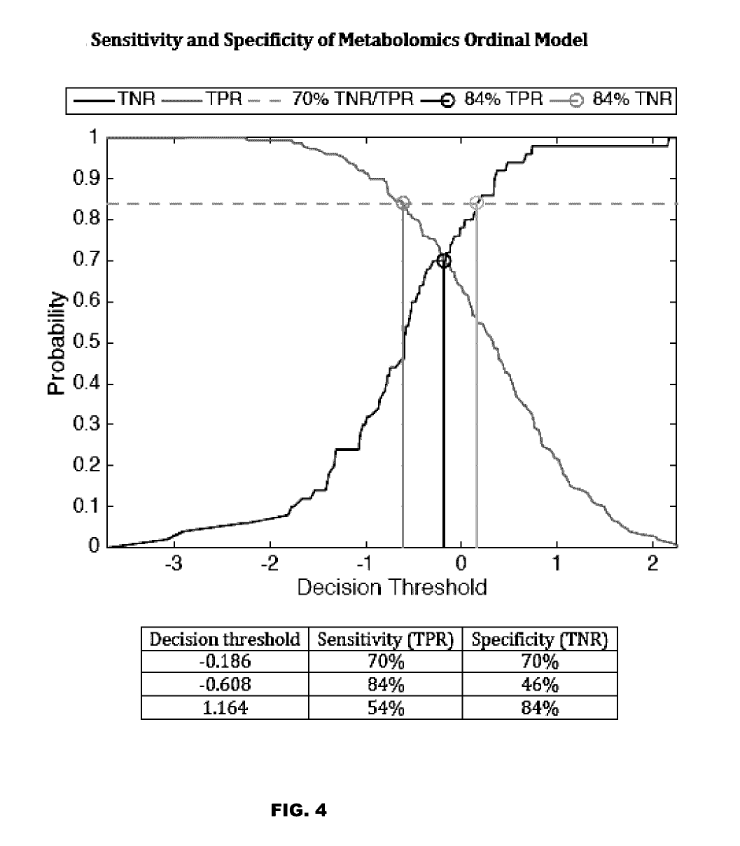

When comparing mild vs. intermediate and advanced comparison (B3) using the ordinal model, a 70% sensitivity and specificity result with AUC 0.7757 was observed (see Table 3). If the sensitivity is set to 84%, the resulting specificity decreases to only 46%. However, if we set the specificity to 84%, the resulting sensitivity is 54.7%. This trade-off of optimizing sensitivity as opposed to specificity is a consequence of our predefined threshold of the output used in our ordinal model. Optimizing sensitivity comes at the cost of decreasing specificity, and vice versa. FIG. 4 depicts the sensitivity and specificity curves as a function of the decision threshold imposed on the ordinal model and the threshold values for the examples illustrated above.

Table 8 lists the top 10 metabolites associated with fibrosis stage after controlling for run day, age, BMI, gender, past/current alcohol use and smoking using multiple logistic regression.

D. Discussion

We report a novel blood-based metabolic profile that correlates with the severity of hepatic fibrosis and may serve has a diagnostic and/or prognostic biomarker for patients with NAFLD and/or NASH. The metabolic profile is robust in its performance; the strength of its association with hepatic fibrosis persisted even after controlling for other metabolic confounding variables such as obesity, diabetes, hyperlipidemia and hypertension. Moreover, the strength of the association of the metabolomics biomarker with fibrosis persisted, albeit less significantly, after controlling for other histologic features of liver injury associated with NASH (i.e., grade of necroinflammation, ballooned hepatocytes, and NAS score). Therefore, the proposed metabolomics profile may also be potentially useful for the diagnosis of NASH as necroinflammation and ballooned hepatocytes are primary predictors for hepatic fibrosis. A large validation study would be necessary to assess whether our biomarker stratifies NAFL from NASH independent of fibrosis. Future longitudinal cohort studies are also needed to define whether the proposed biomarker can predict future progression to advanced hepatic fibrosis or cirrhosis in those with NASH.

Interestingly, the top 10 metabolites found by univariate analysis belong to the superfamily of lipids, vitamin and amino acid pathways. Besides, 4 of the top 10 metabolites belong to the primary bile acid metabolism subfamily. Specifically, taurochenodeoxycholate, glyocholate, glycochenodeoxcholate, and taurocholate were independently and strongly associated with hepatic fibrosis. Despite enriching our cohort with patients with intermediate risk (i.e., stage 2 fibrosis), the association of primary bile acid metabolism with fibrosis severity was notable (see FIG. 1). When compared to patients at low risk for negative clinical outcomes (those patients with little to no hepatic fibrosis) patients with advanced fibrosis had elevated bile acids levels. Bile acids not only play a role in dietary lipid absorption and cholesterol homeostasis, but also serve as signaling molecules for the nuclear hormone receptors, specifically farnesoid X receptors (FXR) and G-protein coupled receptors. In recent studies of patients with hepatitis B-induced cirrhosis and primary biliary cirrhosis, glycocholate (GCA), glycochenodeoxycholate (GCDCA), taurocholate and taurochenodeoxycholate (TCDCA), and glycoursodeoxycholate (GCDCA) were noted to be significantly altered when compared to healthy controls. In support of the notion that alternations in bile acids may serve as a biomarker for advanced hepatic fibrosis, Wang et al. demonstrated that bile acids increase across Child Pugh stages A, B and C. Therefore, alterations in bile acids may not only stratify fibrosis risk but potentially the associated clinical outcomes of advanced liver disease (i.e., diminished liver synthetic function or complications of portal hypertension).

Our study evaluated patients across the spectrum of NAFLD for whom a diagnostic liver biopsy to stage the severity of their liver disease was considered necessary (i.e., patients with no overt clinical, radiographic and/or laboratory features of advanced liver disease as assessed experienced hepatologists). Despite enriching the study population with patients with ≦stage 2 fibrosis (75% of the study cohort), serum bile acids were found to be associated with fibrosis severity. This finding is compelling, provocative, and cannot readily be explained away as all patients in our cohort had normal liver synthetic function and normal serum bilirubin levels. Thus, serum bile acids may not only stage liver cirrhosis across Child-Pugh classes, but define severity hepatic fibrosis prior to clinical signs of hepatic decompensation.

Alternatively, increased serum bile acids in NASH may be a consequence of altered cholesterol metabolism. Although bile acids have many physiological roles, increased levels of bile acids may increase the risk for liver injury due to their association with oxidative stress, inflammation, necrosis, fibrosis and even cirrhosis. Patients with NASH have been shown to have significantly higher total serum bile acid concentrations, including the more hydrophilic and cytotoxic secondary bile acids compared to healthy subjects in both the fasting and fed states, suggesting that increased bile acid exposure may be involved in liver injury and the pathogenesis of NASH. To this end, obeticholic acid, a farnestoid X receptor (FXR) agonist, improved the features of NASH including improvement in hepatic fibrosis in those randomized to obeticholic acid vs placebo (35% vs 19%, p<0.004).

In addition to alterations in bile acids, our study demonstrated increased circulating levels of several TCA cycle intermediates including malate and fumarate (see FIG. 2). Although directionality cannot be discerned from static measurements, increased levels of TCA cycle intermediates has been traditionally associated with decreased mitochondrial oxidative activity and energy generation. Alterations in activity of the mitochondrial respiratory chain may be related to changes in “upstream” metabolic pathways that feed into the TCA cycle, including glycolysis and fatty acid oxidation. Indeed, changes in glucose metabolism were readily apparent in this study. To this end, recent investigations demonstrate that the Hedgehog (Hh) pathway, a key pathway which controls transdifferentiation of hepatic stellate cells, controls the fate of HSCs by regulating metabolism. The transdifferentiation of cultured, quiescent HSCs into myofibroblasts induced glycolysis and caused lactate accumulation. Further, the regulation of glycolysis required Hh signaling and involved the induction of HIF1alpha. Inhibitors of Hh signaling and parallels histologic severity of injury and fibrosis in human NAFLD, HIF1alpha, glycolysis, or lactate accumulation converted myofibroblasts to quiescent HSCs. These finding suggest that changes in glucose metabolism may correlate with severity of fibrosis. The finding of increased circulating levels of TCA cycle intermediates and their association with hepatic fibrosis serve as an applied diagnostic tool to stratify patient with advanced hepatic fibrosis and/or those at risk for fibrosis progression. The role of metabolomics in predicting risk for fibrosis progression will only be defined using large population based longitudinal studies of patients with NAFLD and NASH.

Lastly, our study noted the accumulation of BCAAs and associated metabolites. The accumulation of BCAA and associated metabolites could be related to elevated branched-chain aminotransferase 1 (BCAT1) activity. BCAT1 is elevated in advanced liver disease and its hepatic expression is upregulated in patients with NASH. Because BCAAs are primarily metabolized in mitochondria, changes in BCAAs and related metabolites may result from altered mitochondrial function. Alterations in BCAA could be a novel biomarker for discrimination of metabolic syndrome (38) and potentially even NAFLD. Ferrara et al., using an integrative analysis of metabolite profiling with liver mRNA expression and genomic analysis, demonstrate that glutamate metabolism contributes to chronic complex and highly prevalent disease and conditions such as obesity and diabetes. Thus, the increase in BCAA and associated metabolites in patients with NASH at increased risk for fibrosis progression is plausible. Indeed, our metabolomic analysis revealed an association of BCAA with fibrosis that persisted even after controlling for obesity and T2DM. Batch et al. recently reported that BCAA and related metabolites were noted to distinguish metabolically well from metabolically unwell individuals independent of BMI suggesting that BCAAs are promising biomarkers that may aid in the understanding of cardiometabolic risk independent of BMI.

This study has several strengths. First, our study cohort is well phenotyped for their clinical and histologic features of NAFLD and NASH, which facilitated our ability to compare our metabolomics biomarker against other well-studied non-invasive fibrosis markers. The metabolomics signature demonstrated a stronger AUC compared to AST/ALT ratio, APRI index, FIB-4 score, BARD score, and NAFLD fibrosis scores. Second, the metabolomics analysis was an unbiased analysis of all detected and named metabolites. Third, all serum and/or plasma samples analyzed in our analysis of metabolites were obtained on the same day as liver histology thus making the potential strength of the association robust. Fourth, to our knowledge, this analysis is the largest reported series of metabolomics in NAFLD. Lastly, our study cohort is representative of patients for whom a clinician may otherwise consider a diagnostic liver biopsy (i.e., no overt signs on preexisting cirrhosis) and thus is an ideal cohort for biomarker discovery. Validation of such a biomarker on other independent cohorts is necessary before the introduction of such a biomarker into clinical practice.

The study has a few limitations. First, we acknowledge a population selection bias as all patients analyzed in our cohort had a clinical indication for a standard of care liver biopsy as determined by experienced hepatologists. Therefore, our study cohort may not be representative of patients who otherwise are deemed not to warrant a liver biopsy (i.e., lack of risk factors for NAFLD disease progression or signs/symptoms for advanced liver disease thereby not justifying a liver biopsy for staging). However, this potential weakness may also be considered a strength as our cohort was enriched with high-risk patients for whom a liver biopsy was warranted despite a comprehensive evaluation by a specialists experienced the evaluation and care of patient with NAFLD and NASH. The association of the metabolomics biomarker with the severity of hepatic fibrosis was strong despite this limitation lending credibility that the biomarker would be a reliable predictor of advanced hepatic fibrosis when utilized used in general practice. Second, one may argue that another limitation of our study was the lack of a healthy control cohort. However, we argue that the important and pressing public health need for a biomarker is not to define the presence/absence of NAFLD but to stratify those at risk for advance hepatic fibrosis and associated clinical outcomes. Further, discriminating between isolated hepatic steatosis and steatohepatitis is not clinically impactful, as neither histologic feature, in the absence of advanced hepatic fibrosis, has been associated with increased liver-related morbidity or mortality. However, hepatic fibrosis, and only hepatic fibrosis predicts negative clinical outcomes, thus underscoring the high impact of a blood-based biomarker for stratifying those patients with NASH and advanced hepatic fibrosis.

In conclusion, utilizing a metabolomics analytic approach, we have identified several novel serum and/or plasma biomarkers that are associated with the severity of hepatic fibrosis in NAFLD. We acknowledge that the proposed metabolomics biomarker requires broad validation prior to translation into clinical practice. Such a profile may have both a screening and diagnostic utility in identifying patients at increased risk for advanced hepatic fibrosis or cirrhosis for the purpose of monitoring and surveillance of complications of advanced liver disease. The metabolites noted in this study shed light on NAFLD disease pathogenesis and novel targets for pharmacologic therapies for NASH and NASH-related hepatic fibrosis and cirrhosis.

TABLE 1
Patient Characteristics

| | Fibrosis stage | | | | | | |
|---|---|---|---|---|---|---|---|
| | | Mild | | Intermediate | Advanced | | |
| | All | 0 | 1 | 2 | 3 | 4 | |
| | N = 200 | N = 12 | N = 38 | N = 100 | N = 42 | N = 8 | p-value |
| Age (years) | 50.3 ± 10.5 | 53.4 | 48.9 | 49.6 | 50.7 | 59.1 | 0.305 |
| Sex (male, %) | 47.5 | 41.7 | 42.1 | 50.0 | 52.4 | 25.0 | 0.765 |
| BMI | 35.0 ± 7.2 | 34.4 | 34.7 | 34.9 | 34.9 | 38.5 | 0.384 |
| Diabetes mellitus (%) | 37.5 | 16.7 | 21.1 | 36.0 | 54.8 | 75.0 | 4.9 × 10⁻⁵ |
| Hypertension (%) | 64.5 | 50.0 | 52.6 | 64.0 | 78.8 | 75.0 | 9.8 × 10⁻³ |
| Hyperlipidemia (%) | 38.0 | 25.0 | 28.9 | 38.0 | 50.0 | 37.5 | 0.061 |
| Hypercholesterolemia | 29.0 | 41.7 | 36.8 | 26.0 | 26.2 | 25.0 | 0.174 |
| (%) | | | | | | | |
| Hypertriglyceridemia (%) | 15.0 | 16.7 | 15.8 | 12.0 | 19.0 | 25.0 | 0.567 |
| Current alcohol (%) | 46.0 | 50.0 | 57.9 | 41.0 | 47.6 | 37.5 | 0.331 |
| Past alcohol (%) | 51.0 | 50.0 | 60.5 | 44.0 | 59.5 | 50.0 | 0.995 |
| Current smoking (%) | 12.5 | 16.7 | 18.4 | 8.0 | 14.3 | 25.0 | 0.884 |
| Past smoking (%) | 36.0 | 25.0 | 50.0 | 29.0 | 40.5 | 50.0 | 0.855 |
| Triglycerides (mg/dl) | 184.2 ± 138.3 | 224.0 | 154.0 | 183.8 | 204.7 | 163.8 | 0.679 |
| Total cholesterol (mg/dl) | 192.3 ± 48.1 | 189.6 | 196.2 | 187.2 | 202.3 | 187.4 | 0.675 |
| HDL-cholesterol (mg/dl) | 41.0 ± 16.6 | 41.1 | 45.6 | 40.1 | 39.6 | 36.5 | 0.143 |
| LDL-cholesterol (mg/dl) | 114.6 ± 33.8 | 113.8 | 118.5 | 112.1 | 116.5 | 118.1 | 0.982 |
| AST (U/L) | 67.5 ± 47.8 | 33.6 | 61.7 | 71.1 | 73.7 | 67.4 | 0.032 |
| ALT (U/L) | 86.3 ± 61.4 | 48.4 | 85.3 | 91.4 | 88.2 | 73.1 | 0.294 |
| Alkaline phosphatase | 88.7 ± 52.3 | 87.1 | 82.8 | 91.0 | 90.9 | 77.5 | 0.791 |
| (U/L) | | | | | | | |
| Total bilirubin (mg/dl) | 0.8 ± 0.3 | 0.6 | 0.7 | 0.8 | 0.8 | 0.7 | 0.029 |
| Platelet count | 236.2 ± 71.0 | 274.6 | 258.1 | 231.3 | 226.2 | 187.8 | 8.9 × 10⁻⁴ |
| (thou/cumm³) | | | | | | | |
| HbA1C (%) | 6.3 ± 1.2 | 5.8 | 6.2 | 6.3 | 6.8 | 6.8 | 2.4 × 10⁻³ |
| Steatosis grade | 1.8 ± 0.8 | 1.5 ± 0.9 | 1.9 ± 0.8 | 1.8 ± 0.8 | 1.8 ± 0.8 | 1.6 ± 0.9 | 0.755 |
| Lobular inflammation | 1.3 ± 0.6 | 0.8 ± 0.4 | 1.3 ± 0.6 | 1.3 ± 0.5 | 1.5 ± 0.7 | 1.4 ± 0.5 | 1.3 ± 10⁻³ |
| Portal inflammation | 0.4 ± 0.5 | 0.1 ± 0.3 | 0.2 ± 0.4 | 0.4 ± 0.5 | 0.5 ± 0.5 | 0.9 ± 0.4 | 9.6 ± 10⁻⁶ |
| Hepatacellular ballooning | 1.2 ± 0.7 | 0.4 ± 0.5 | 1.1 ± 0.7 | 1.2 ± 0.6 | 1.5 ± 0.6 | 1.5 ± 0.8 | 2.9 ± 10⁻⁷ |
| NAS Score | 4.1 ± 1.8 | 2.8 ± 1.3 | 4.1 ± 1.6 | 4.0 ± 1.9 | 4.7 ± 1.7 | 3.8 ± 2.1 | 0.014 |

Values expressed as mean ± standard deviation.
Abbreviations:
BMI = body mass index.
HDL = high density lipoprotein;
LDL = low density lipoprotein;
AST = aspartate aminotransferase;
ALT = alanine aminotransferase;
HbA1c = glycosylated hemoglobin;
NAS = nonalcoholic steatohepatitis activity score.

TABLE 2
Univariate Analysis

| | Pathway | | p-value (FDR) | | |
|---|---|---|---|---|---|
| Metabolite | Super | Sub | M1 | M2 | M3 |
| 1-urobilinogen | Cofactors and | Hemoglobin and Porphyrin | 0.0022 | 0.0018 | 0.0149 |
| | Vitamins | Metabolism | | | |
| taurochenodeoxycholate | Lipid | Primary Bile Acid | 0.0027 | 0.0076 | 0.0149 |
| | | Metabolism | | | |
| glycocholate | Lipid | Primary Bile Acid | 0.0071 | 0.0105 | 0.0328 |
| | | Metabolism | | | |
| glycochenodeoxycholate | Lipid | Primary Bile Acid | 0.0071 | 0.0115 | 0.0328 |
| | | Metabolism | | | |
| malate | Energy | TCA Cycle | 0.0081 | 0.0146 | 0.1010 |
| taurocholate | Lipid | Primary Bile Acid | 0.0081 | 0.0150 | 0.0328 |
| | | Metabolism | | | |
| glutarate | Amino Acid | Lysine Metabolism | 0.0159 | 0.0146 | 0.0848 |
| (pentanedioate) | | | | | |
| fumarate | Energy | TCA Cycle | 0.0201 | 0.0321 | 0.1309 |
| 1-arachidonoyl-GPI* | Lipid | Lysolipid | 0.0645 | 0.0234 | 0.0328 |
| (20:4)* | | | | | |
| 3-ureidopropionate | Nucleotide | Pyrimidine Metabolism; | 0.0912 | 0.0234 | 0.0767 |
| | | Uracil containing | | | |
| 16a-hydroxy DHEA 3- | Lipid | Steroid | 0.0492 | 0.0285 | 0.1197 |
| sulfate | | | | | |
| 1-stearoyl-GPI (18:0) | Lipid | Lysolipid | 0.0492 | 0.0311 | 0.1197 |
| 3- | Amino Acid | Lysine Metabolism | 0.1157 | 0.0386 | 0.1008 |
| methylglutarylcarnitine | | | | | |
| (2) | | | | | |
| gamma- | Peptide | Gamma-glutamyl Amino | 0.0410 | 0.0458 | 0.1395 |
| glutamylisoleucine* | | Acid | | | |
| glutamate | Amino Acid | Glutamate Metabolism | 0.0912 | 0.0458 | 0.2093 |

M1 only controls for batch effects (run day).
M2, same as M1 plus age, gender, BMI, past/current alcohol and past/current smoking.
M3, same as M2 plus diabetes mellitus and hypertension.
M4, same as M3 plus Steatosis, lobular inflammation, portal inflammation, and hepatocyte ballooning, no significant differences after correction for multiple testing (in supplemental information).

TABLE 3
Multivariate Analysis

| | Fibrosis Stage Comparison AUCs | | |
|---|---|---|---|
| | B1: Mild vs. | B2: Mild and | B3: Mild |
| | Advanced | Intermediate | vs. Intermediate |
| | Stage 0, 1 | vs. Advanced | and Advanced |
| Model | Vs. 3, 4 | 0, 1, 2 Vs. 3, 4 | 0, 1 Vs. 2, 3, 4 |
| AST/ALT ratio | 0.6294 | 0.6005 | 0.5629 |
| Bard score | 0.6688 | 0.6118 | 0.6265 |
| APRI index | 0.7418 | 0.6438 | 0.6843 |
| Fib4 score | 0.7748 | 0.6772 | 0.6794 |
| NAFLD fibrosis score | 0.7280 | 0.6573 | 0.6732 |
| NAFLD clinical score | 0.6896 | 0.6137 | 0.6373 |
| Metabolites binary | 0.8228 | 0.7220 | 0.7293 |
| Metabolites ordinal | 0.8636 | 0.7216 | 0.7757 |

If we restrict the comparison to match, AUCs are: 0.7517, 0.6724 and 0.6830 for B1, B2, and B3, respectively.

TABLE 4
Clinical Patient Characteristics

| | | | Inter- | | | |
|---|---|---|---|---|---|---|
| | Mild | | mediate | Advanced | | |
| | Fibrosis | | Fibrosis | Fibrosis | | |
| | (n = 50) | | (n = 100) | (n = 50) | | |
| Number of | Stage 0 | Stage 1 | Stage 2 | Stage 3 | Stage 4 | p- |
| Samples | (n = 12) | (n = 38) | (n = 100) | (n = 42) | (n = 8) | value |
| Age (yrs) | 53 ± 9 | 49 ± 11 | 50 ± 11 | 51 ± 8 | 59 ± 5 | 0.095 |
| Gender | 5 (42%) | 16 (42%) | 50 (50%) | 22 (52%) | 2 (25%) | 0.584 |
| (M) | | | | | | |
| BMI | 34 ± 8 | 35 ± 7 | 35 ± 7 | 35 ± 7 | 38 ± 14 | 0.731 |
| (kg/m²) | | | | | | |
| Diabetes | 2 (17%) | 8 (21%) | 36 (36%) | 23 (55%) | 6 (75%) | 0.019 |

Mean ± SD
No difference between groups in race, past or present smoking or alcohol use, AST, ALT, total-, HDL-, LDL-cholesterol or triglycerides.

TABLE 5
Histologic Patient Characteristics

| | Mild | | Intermediate | Advanced | | |
|---|---|---|---|---|---|---|
| | Fibrosis | | Fibrosis | Fibrosis | | |
| | (n = 50) | | (n = 100) | (n = 50) | | |
| | Stage 0 | Stage 1 | Stage 2 | Stage 3 | Stage 4 | |
| Number of Samples | (n = 12) | (n = 38) | (n = 100) | (n = 42) | (n = 8) | p-value |
| Steatosis | 1.5 ± 0.9 | 1.9 ± 0.8 | 1.8 ± 0.8 | 1.8 ± 0.8 | 1.6 ± 0.9 | 0.662 |
| Lobular Inflammation | 0.8 ± 0.4 | 1.3 ± 0.6 | 1.4 ± 0.6 | 1.6 ± 0.7 | 1.4 ± 0.5 | 0.005 |
| Portal Inflammation | 0.1 ± 0.3 | 0.2 ± 0.4 | 0.4 ± 0.5 | 0.5 ± 0.5 | 1.0 ± 0.0 | 0.0001 |
| Ballooning | 0.4 ± 0.5 | 1.1 ± 0.7 | 1.2 ± 0.6 | 1.5 ± 0.6 | 1.5 ± 0.8 | <0.0001 |
| NAS | 2.8 ± 1.3 | 4.2 ± 1.5 | 4.5 ± 1.4 | 5.0 ± 1.3 | 4.3 ± 1.6 | 0.0001 |

Mean ± SD. Pathology components graded/staged according to NASH CRN criteria.
Portal Inflammation grade: 0 = absent, 1 = present

TABLE 6

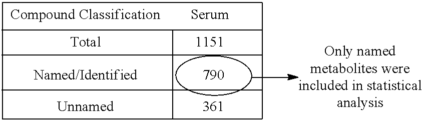

TABLE 7
Univariate Analysis
Association of metabolites with fibrosis stage normalized for run day only.
Top 10 of 790 metabolites

| Metabolite | Super Family | Sub Family | P-value | FDR |
|---|---|---|---|---|
| I-urobilinogen | Cofactors and | Hemoglobin/Porphyrin | 3.31e−06 | 0.002 |
| | Vitamins | Metabolism | | |
| taurochenodeoxycholate | Lipid | Primary Bile Acid Metabolism | 7.98e−06 | 0.003 |
| glycocholate | Lipid | Primary Bile Acid Metabolism | 4.18e−5 | 0.007 |
| glycochenodeoxycholate | Lipid | Primary Bile Acid Metabolism | 3.61e−5 | 0.007 |
| malate | Energy | TCA Cycle | 6.81e−5 | 0.008 |
| taurocholate | Lipid | Primary Bile Acid Metabolism | 7.16e−5 | 0.008 |
| glutarate | Amino Acid | Lysine Metabolism | 0.0002 | 0.016 |
| (pentanedioate) | | | | |
| fumarate | Energy | TCP Cycle | 0.0002 | 0.020 |
| Gamma- | Peptide | Gammy-glutamyl amino acid | 0.0005 | 0.041 |
| glutamylisoleucine | | | | |
| 3-hydroxyoctanoate | Lipid | Fatty Acid, monohydroxy | 0.0006 | 0.049 |

TABLE 8
Multiple Logistic Regression
Association of metabolites with fibrosis stage after controlling
for run day, age, BMI, gender, past/current alcohol use and smoking:

| Metabolite | Super Family | Sub Family | P-value | FDR |
|---|---|---|---|---|
| I-urobilinogen | Cofactors and | Hemoglobin and Porphyrin | 2.75e−06 | 0.002 |
| | Vitamins | Metabolism | | |
| taurochenodeoxycholate | Lipid | Primary Bile Acid Metabolism | 2.24e−05 | 0.008 |
| glycocholate | Lipid | Primary Bile Acid Metabolism | 4.62e−5 | 0.011 |
| glycochenodeoxycholate | Lipid | Primary Bile Acid Metabolism | 6.76e−5 | 0.011 |
| glutarate (pentanedioate) | Amino Acid | Lysine Metabolism | 0.0001 | 0.002 |
| malate | Energy | TCA Cycle | 0.0001 | 0.015 |
| taurocholate | Lipid | Primary Bile Acid Metabolism | 0.0002 | 0.015 |
| 1-arachidonoyl-GPI* (20:4) | Lipid | Lysolipid | 0.0004 | 0.023 |
| 3-ureidopropionate | 3′Nucleotide | Pyrimidine Metabolism | 0.0002 | 0.023 |
| 16a-hydroxy DHEA 3- | Lipid | Steroid | 0.0004 | 0.029 |
| sulfate′ | | | | |

4 of top 10 metabolites associated with fibrosis are of the family of primary bile acid metabolism.

TABLE 9
Multiple Logistic Regression
Association of metabolites with fibrosis stage after controlling for run day, clinical
factors, PLUS diabetes mellitus (DM), pharmacology therapy for DM and hypertension:

| Metabolite | Super Family | Sub Family | P-value | FDR |
|---|---|---|---|---|
| taurochenodeoxycholate | Lipid | Primary Bile Acid | 3.60e−05 | 0.015 |
| | | Metabolism | | |
| I-urobilinogen | Cofactors and | Hemoglobin and Porphyrin | 4.39e−05 | 0.015 |
| | Vitamins | Metabolism | | |
| 1-arachidonoyl-GPI* (20:4) | Lipid | Lysolipid | 0.0003 | 0.033 |
| glycocholate | Lipid | Primary Bile Acid Metabolism | 0.0002 | 0.033 |
| taurocholate | Lipid | Primary Bile Acid Metabolism | 0.0002 | 0.033 |
| glycochenodeoxycholate | Lipid | Primary Bile Acid Metabolism | 0.0002 | 0.033 |
| epiandrosterone sulfate | Lipid | Steroid | 0.0009 | 0.077 |
| 3-ureidopropionate | 3′Nucleotide | Pyrimidine Metabolism | 0.0009 | 0.077 |
| glutarate (pentanedioate) | Amino Acid | Lysine Metabolism | 0.0011 | 0.066 |
| 3-methylglutarylcarnitine | Amino Acid | Lysine Metabolism | 0.0015 | 0.101 |

Metabolites of primary bile acid remained strongly associated with fibrosis independent of clinical factors.

### REFERENCES

Any patents or publications mentioned in this specification are indicative of the levels of those skilled in the art to which the invention pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present invention is well adapted to carry out the objects and obtain the ends and advantages mentioned, as well as those inherent therein.

1. A panel of non-invasive metabolomic biomarkers for nonalcoholic fatty liver disease (NAFLD) comprising at least two, at least 3, at least 4, or at least 10 metabolomic biomarkers listed in any of Table 2, Table 7, Table 8 or Table 9 which are associated with NFLD.

2. The panel of claim 1, wherein expression of the panel of metabolomic biomarkers is associated with liver fibrosis.

3. The panel of claim 1, wherein the panel of metabolomic biomarkers comprises the metabolomic biomarkers listed in TABLE 2.

4. The panel of claim 1, wherein the panel comprises the metabolomic biomarkers listed in TABLE 7.

5. The panel of claim 1, wherein the panel comprises metabolomic biomarkers listed in TABLE 8.

6. The panel of claim 1 wherein the panel comprises metabolomic biomarkers listed in TABLE 9.

7. The panel of claim 1, wherein the panel comprises taurochenodeoxycholate, glyocholate, glycochenodeoxcholate, and taurocholate, wherein the expression of the panel is associated with advanced hepatic fibrosis (stage 3-4).

8. The panel of claim 1, wherein the panel comprises glutamate, 3 methlglutarylcarintine and glutamate, and wherein the expression of the panel is associated with advanced hepatic fibrosis.

9. A method of detecting a panel of metabolomics biomarkers associated with nonalcoholic fatty liver disease or liver fibrosis in a subject comprising:
obtaining a sample from a subject;
detecting at least two, at least 3, at least 4, or at least 10 non-invasive metabolite fibrosis markers listed in any of Tables 2, 7, 8, or 9 in a sample obtained from the subject; wherein the detection of at least two markers is associated with NAFLD or liver fibrosis.

10. The method of claim 9, wherein the sample is a serum sample or blood sample.

11. The method of claim 9, wherein expression of at least three, at least four, at least five, at least six, at least seven, at least eight, at least nine, at least 10, at least 11, at least 12, at least 13, at least 14 metabolomics biomarkers are detected.

12. The method of claim 9, wherein the detection of the panel indicates the presence of liver fibrosis.

13. The method of claim 12, wherein an increase level of the metabolite marker indicated an increased severity of liver fibrosis.

14. The method of claim 9, wherein the method further comprises diagnosing the patient with liver fibrosis.

15. The method of claim 14, wherein the method further comprises determining an increased level of the at least two non-invasive metabolite fibrosis markers, wherein increased levels of the markers indicates a more advanced level of fibrosis.

16. The method of claim 14, wherein the method further comprises: treating the subject diagnosed or prognosed with liver fibrosis.

17. The method of claim 16, wherein treating the subject comprises treating the fibrosis by administering an anti-fibrotic therapy to the subject diagnosed or prognosed with liver fibrosis.

18. The method of claim 9, wherein the metabolite markers comprise taurochenodeoxycholate, glyocholate, glycochenodeoxcholate, and taurocholate, and wherein the expression of the panel is associated with advanced hepatic fibrosis (stage 3-4).

19. The method of claim 9, wherein the metabolite markers comprise glutamate, 3 methlglutarylcarintine and glutamate or malate and fumarate, and wherein the expression of the panel is associated with advanced hepatic fibrosis.

20. A kit for detecting NAFLD or liver fibrosis in a subject comprising means for detecting at least two, at least 3, at least 4, or at least 10 metabolomic biomarkers in a sample.
